(19) United States
(12) Patent Application Publication
(10) Pub. No.: US 2022/0409417 A1
(43) Pub. Date: Dec. 29, 2022

(54) Adjustable Therapeutic Scoliosis Brace

(71) Applicant: Stephen Barr, Cave Creek, AZ (US)

(72) Inventor: Stephen Barr, Cave Creek, AZ (US)

(21) Appl. No.: 17/357,268

(22) Filed: Jun. 24, 2021

(51) Int. Cl. A61F 5/02; A61F 5/34

(52) U.S. Cl. CPC A61F 5/024; A61F 5/34

(57) ABSTRACT

The present invention relates to an adjustable scoliosis correction brace that presents a novel structure of scoliosis correction brace with multiple inflatable airbags and are placed at multiple places. These bags can be inflated with air to presses against that curve where the air can be measured with an air gauge attached to the pump which will measure the amount of pressure being applied and trains the spine to return to a straighter portion, reducing the scoliotic curve. Once required to be deflated the air pressure hose can be removed and the brace worn as a normal brace would be worn.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention is generally directed to the field of orthotics and in particular relates to a scoliosis correction brace that uses air pressure which is measured by air gauge to put additional pressure on the scoliosis curve and help with the correction.

### Description of the Related Art

Scoliosis is a medical condition associated with an abnormally curved spine, often thought to be a progressive disease, at least until adulthood. Scoliosis can have severe adverse effects on a patient's life, both physically and physiologically. One possible method of treating or managing scoliosis is surgery. Unfortunately, scoliosis surgery can be very risky. Less invasive methods of treating or managing scoliosis has traditionally included physical therapy, chiropractic therapy, or bracing, among other things.

Some previous efforts have been made to provide improved comfort, support, or customizability to individual wearers. For instance, U.S. Pat. No. 170,655 describes a back and shoulder brace. This brace appears to have a single shoulder brace M, and the hip supports A, A1 appear to be disposed to the front and back of the wearer, and not under the arms. It does not appear that hip supports A, A1 can be adjusted in length.

U.S. Pat. No. 492,903 shows a flexible brace for curing spinal curvature which relies on lateral forces. The steel supports which extend up from the pelvis belt are disposed at the back, and do not directly support the shoulders.

U.S. Pat. Nos. 970,781 and 1,595,739 also appear to be intended for treating spinal curvature, and do not have rigid shoulder supports under both arms of the wearer.

U.S. Pat. No. 2,687,129 describes a corrective brace for a scoliosis patient. This device includes a single rigid hip pad, a chest pad, supporting straps carried by the hip and chest pads, a tension strap and aligned connecting bars disposed under one arm and secured to the chest and hip pads. The purpose seems to be to straighten a crooked spine by applying lateral compression.

U.S. Pat. No. 3,029,810 discloses a somewhat similar back brace which has a single adjustable strut which is disposed under one arm of the patient. It also has a pair of arm encircling connected by a shoulder strap. This brace also appears to be intended to give lateral support.

U.S. Pat. No. 3,878,841 shows an adjustable orthotic brace which has a single adjustable support extending from the side of the pelvis up to the armpit of a patient. A cushioned half crutch is connected to the upper end of the adjustable support. A harness holds the crutch in place to support one arm of the patient. A harness is provided to extend across the back of the user, and around the other shoulder to hold the half crutch in place. Each of the above devices provide rigid support only on one side of the user's body.

There are multiple solutions that have been presented in prior art. However, these solutions are limited and restricted to their conventional architecture, installation system and have considerable shortcomings which adversely affect the convenience with which they can be used.

It has to be noted that the current invention proposes an assembly with an advancement where a scoliosis correction brace is presented, that uses air pressure to change the degree of the scoliosis curve over time. The air-bladders inside the brace at the convexity of the curve, will inflate those air-bladders that will put additional pressure on the scoliosis curve and help with the correction. The amount of pressure measured with an air gauge attached to the pump will measure the amount of pressure being applied.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of an improved form of the scoliosis correction brace with inflatable air bladders.

Another object of the present invention is to provide an advanced mechanism of a scoliosis correction brace that uses air pressure to change the degree of the scoliosis curve over time.

Another object of this invention is to provide a new and improved method of a built-in small air bags placed in specific places especially at the convexity of the curve and once these bags are inflated, it presses against that curve and trains the spine to return to a straighter portion, reducing the scoliotic curve.

It is an object of the invention in one of its aspects to provide an assembly which will give supplemental support for the spine and prevent further damage thereto and, in cases of impairment of the spine, to provide helpful relief from pain.

It is also the objective of the invention to provide air gauge attached to the pump will measure the amount of pressure being applied.

It is also the objective of invention to provide variable thickness sizes of the air bladder which will be replaced with a larger size as more correction is being made and will be attached inside using hook and loop fasteners.

Yet another object of the present invention is to provide a structure that is relatively inexpensive to produce so that ownership is available to a substantial majority of families.

It is a further aspect of the present invention to provide a new and improved assembly which is of a durable and reliable construction and can sustain its effectiveness after multiple and consistent use.

Thus, it is the objective to provide a new and improved adjustable Therapeutic Scoliosis Brace with additional advancement of inflation and deflation. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

FIG. 1 discloses front view of adjustable scoliosis correction brace as per preferred embodiments of the invention.

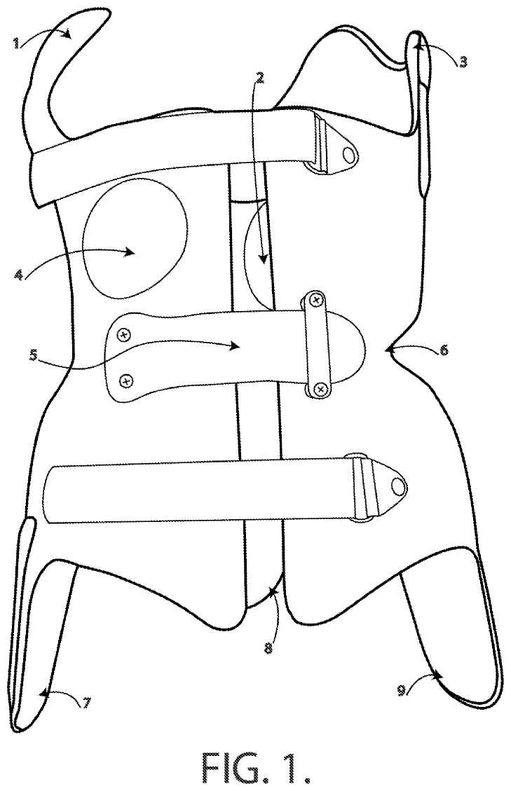

FIG. 2 discloses the air valve air gauge attached to the pump to measure the amount of pressure being applied as per preferred embodiments of the invention.

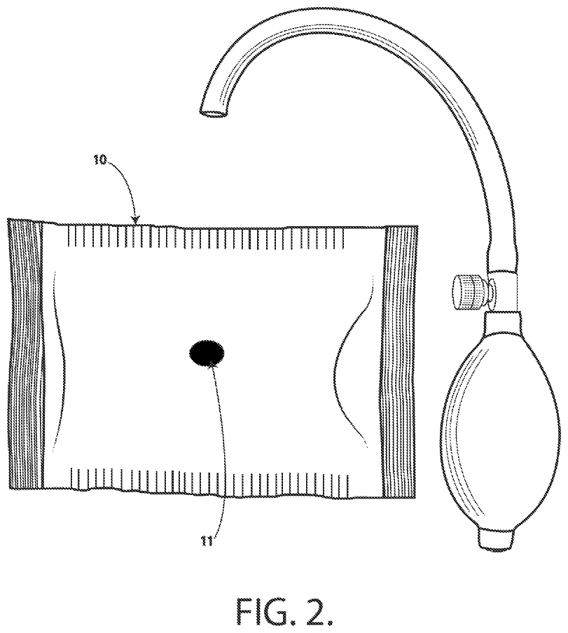

FIG. 3 discloses another view of adjustable scoliosis correction brace as per preferred embodiments of the invention.

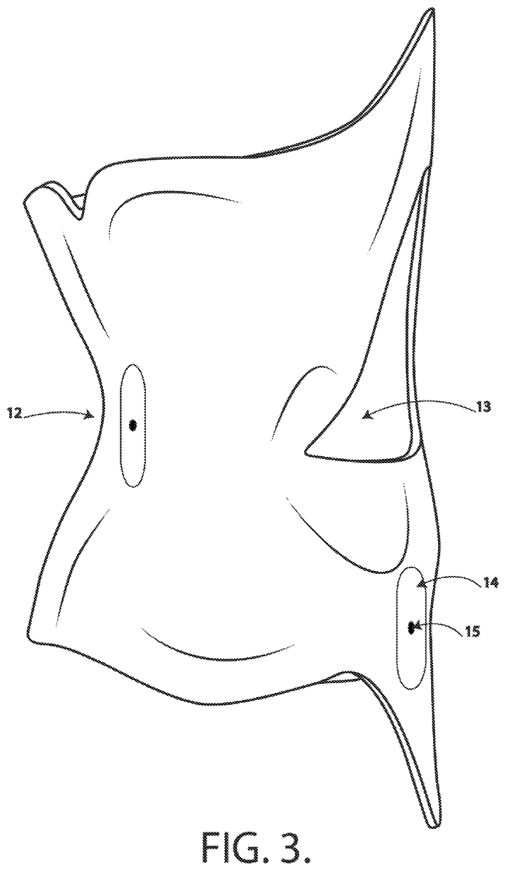

FIG. 4 discloses the use of Hook and loop fasteners allowing the adjustable scoliosis correction brace adjusted as required as per preferred embodiments of the invention.

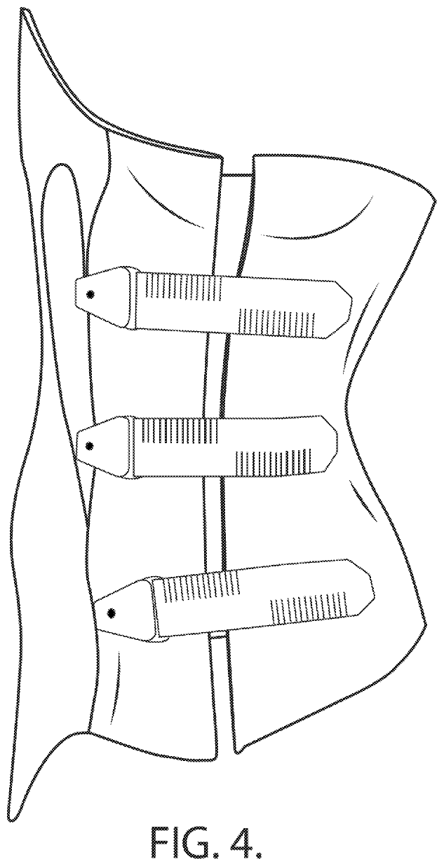

FIG. 5 discloses another view of adjustable scoliosis correction brace as per preferred embodiments of the invention.

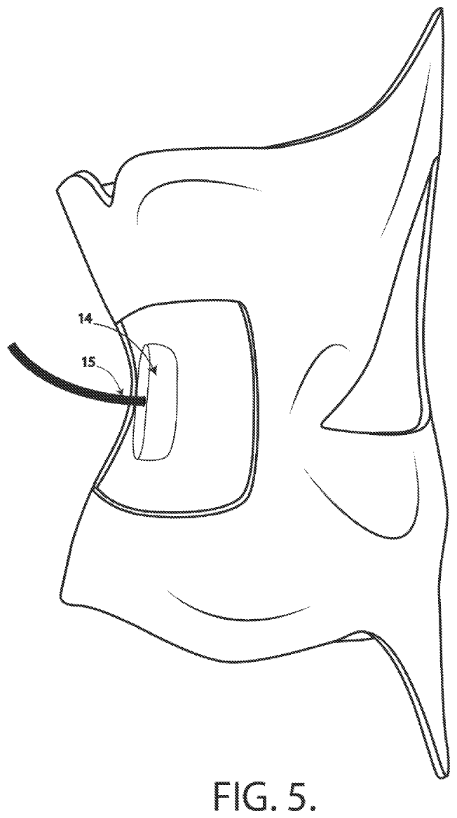

FIG. 6 discloses expansion area of the brace which will allow for the part of the back to move with the correction and give it space to move as per preferred embodiments of the invention.

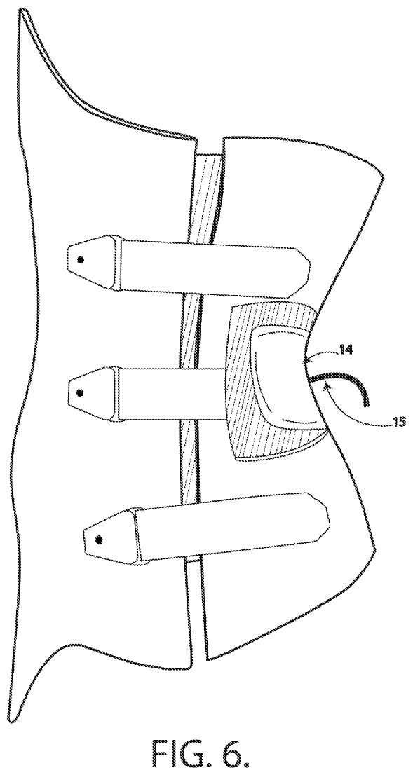

FIG. 7 discloses back view of adjustable scoliosis correction brace as per preferred embodiments of the invention.

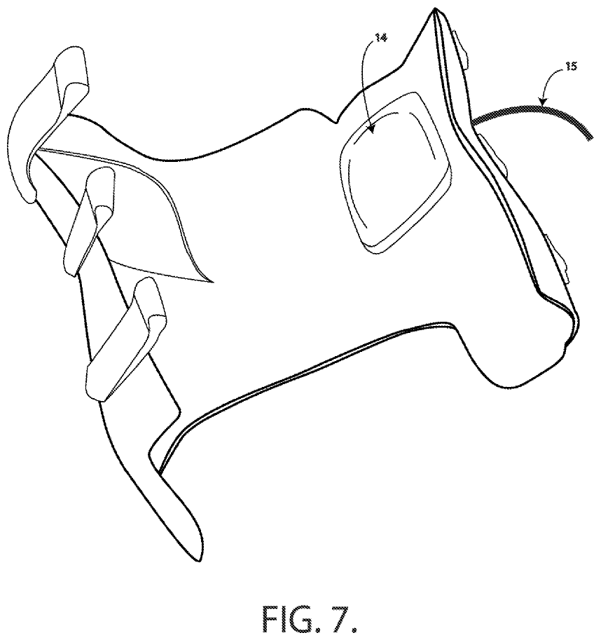

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provide an improved scoliosis correction brace that uses air pressure to change the degree of the scoliosis curve over time.

The assembly as per its preferred embodiments provides a supporting structure comprising a front and back side which is worn by the patient to provide support to accurately rectify the changed contours of the lower body as it follows a corrected flat back position of the lower body.

The present invention as per its further embodiments relates especially to an adjustable, modular brace for recumbent treatment of scoliosis including the use of flexible lateral shells curved and connected together to cover much of the patient's bodice, extending from the hips to the arm pits. The shells are available in a range of types and sizes, which can be combined variously to suit each patient's individual needs.

The inner adjustable therapeutic scoliosis brace is mounted with small airbags placed at specific locations on anterior upright and posterior positions, however mostly at the convexity of the curve. These adjustment and flexible airbags allow the user to selectively select the airbag placed at the anterior and/or posterior positions and allows for adjustment to focus on designated points. The orthopedic apparatus applies a force opposite the lateral curvature of the spine of a scoliosis patient by forcing the patient to bend in a direction opposite to the patient's spinal curvature.

The adjustable therapeutic scoliosis brace as per its preferred embodiments allows placing the air-bladders inside the brace at the convexity of the curve, which will inflate those air-bladders that will put additional pressure on the scoliosis curve and help with the correction. The amount of pressure measured with an air gauge attached to the pump will measure the amount of pressure being applied. The different thickness sizes of the air bladder (⅛″, ¼″, etc.) will be replaced with a larger size as more correction is being made. When the air bladder is fitted to the inside of the Adjustable Therapeutic Scoliosis Brace, the practitioner has the ability to increase pressure at any time in a therapeutic manner. By adding pressure, the air bladder inflates and presses against the convexity of the curve, resulting in faster correction of the scoliosis.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. An adjustable therapeutic scoliosis brace for treatment of scoliosis comprising:
an anterior upright means formed as a unitary structure;
a posterior upright means formed as a unitary structure;
a plurality of airbags at anterior and posterior portions;
the adjustable therapeutic scoliosis brace as per claim 1, which has plurality of air bags placed at multiple selected positions, specifically at convexity of the curve;
the adjustable therapeutic scoliosis brace as per claim 1, where the bags can be inflated presses against that curve and trains the spine to return to a straighter portion, reducing the scoliotic curve;
the adjustable therapeutic scoliosis brace as per claim 1, where the air pressure hose can be removed and the brace can be worn as a normal brace is worn.
the adjustable therapeutic scoliosis brace as per claim 1, where the air bladder (⅛″, ¼″, etc.) can be replaced with a larger size as more correction is being made and can be attached inside using hook and loop fasteners.
